(19) United States
(12) Patent Application Publication
(10) Pub. No.: US 2022/0101965 A1
(43) Pub. Date: Mar. 31, 2022

(54) STORAGE MEDIUM AND PATIENT INFORMATION MANAGEMENT DEVICE

(71) Applicant: Konica Minolta, Inc., Tokyo (JP)

(72) Inventor: Youko Kouda, Tokyo (JP)

(73) Assignee: Konica Minolta, Inc., Tokyo (JP)

(21) Appl. No.: 17/487,341

(22) Filed: Sep. 28, 2021

(30) Foreign Application Priority Data
Sep. 30, 2020 (JP) 2020-164789

(51) Int. Cl. G16H 10/60; G06F 21/62; H04L 29/06

(52) U.S. Cl. CPC G16H 10/60; H04L 63/0876; H04L 63/0853; G06F 21/6245

(57) ABSTRACT

A non-transitory computer readable storage medium stores instructions that cause a computer to execute: confirming whether at least part of first patient information matches at least part of second patient information, wherein the first patient information is obtained from a user terminal used by a patient and the second patient information is obtained from a medical facility; notifying the user terminal of authentication information of the patient, wherein the authentication information is generated upon confirming that the at least part of the first patient information matches the at least part of the second patient information; and associating the first patient information with the second patient information upon reception of related authentication information relating to the authentication information from the user terminal to which the authentication information has been notified.

### CROSS-REFERENCE TO RELATED APPLICATIONS

The entire disclosure of Japanese Patent Application No. 2020-164789 filed on Sep. 30, 2020 is incorporated herein by reference.

### BACKGROUND

### Technical Field

The present invention relates to a storage medium and a patient information management device.

### Description of the Related Art

An application (hereinafter, referred to as a patient application) that enables sharing patient information such as test results obtained in a medical facility with the patient has been conventionally known. In order to use the patient application, identity verification needs to be performed for a registrant of the patient application, and face-to-face identity verification is performed at the medical facility. Thus, when the patient wishes to register for the patient application after once going away from the medical facility, for example, going home from the medical facility, the patient needs to visit the medical facility again and perform face-to-face identity verification, which is troublesome.

Thus, there is proposed, as a method for performing user authentication without face-to-face contact, a method of receiving a phone number assigned to a mobile terminal, transmitting information for verification to the phone number, and in response to receiving a response using the information for verification from the mobile terminal, executing user authentication of the mobile terminal on the basis of the response (for example, see JP 2017-157009 A).

However, in the above method described in JP 2017-157009 A, it is not possible to identify the patient that is the user at the time of registration for the above patient application. Thus, the user cannot be associated with the patient information held by the medical facility, and the patient information regarding the user cannot be provided.

### SUMMARY

One or more embodiments of the present invention provide a storage medium storing instructions and a patient information management device that enable identity verification without face-to-face contact.

According to one or more embodiments of the present invention, a non-transitory computer readable storage medium stores instructions that cause a computer to execute: confirming that is confirming whether at least part of first patient information which is obtained from a user terminal used by a patient matches at least part of second patient information which is obtained from a medical facility; notifying that is notifying the user terminal of authentication information of the patient, the authentication information being generated in response to confirming that the at least part of the first patient information matches the at least part of the second patient information in the confirming; and associating that is associating the first patient information with the second patient information in response to reception of related authentication information which is related to the authentication information from the user terminal that receives the authentication information by the notifying.

According to one or more embodiments of the present invention, a non-transitory computer readable storage medium stores instructions that cause a computer to execute: first obtaining that is obtaining first patient information from a user terminal which is used by a patient; second obtaining that is obtaining second patient information from a medical facility; confirming that is confirming whether at least part of the first patient information matches at least part of the second patient information; generating that is generating authentication information of the patient in response to confirming that the at least part of the first patient information matches the at least part of the second patient information in the confirming; determining that is determining transmission information for transmitting the authentication information to the user terminal; transmitting that is transmitting the authentication information to the user terminal by using the transmission information which is determined in the determining; and associating that is associating the first patient information with the second patient information in response to reception of related authentication information which is related to the authentication information from the user terminal.

According to one or more embodiments of the present invention, a patient information management device includes a hardware processor that: confirms whether at least part of first patient information which is obtained from a user terminal used by a patient matches at least part of second patient information which is obtained from a medical facility; notifies the user terminal of authentication information of the patient, the authentication information being generated in response to confirming that the at least part of the first patient information matches the at least part of the second patient information; and associates the first patient information with the second patient information in response to reception of related authentication information which is related to the authentication information from the user terminal that receives the authentication information.

According to one or more embodiments of the present invention, a patient information management device includes a hardware processor that: obtains first patient information from a user terminal which is used by a patient; obtains second patient information from a medical facility; confirms whether at least part of the first patient information matches at least part of the second patient information; generates authentication information of the patient in response to confirming that the at least part of the first patient information matches the at least part of the second patient information; determines transmission information for transmitting the authentication information to the user terminal; transmits the authentication information to the user terminal by using the determined transmission information; and associates the first patient information with the second patient information in response to reception of related authentication information which is related to the authentication information from the user terminal.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic configuration diagram showing a medical data sharing system;

FIG. 2 is a block diagram showing a functional configuration of a medical system;

FIG. 3 is a block diagram showing a functional configuration of a patient information management server;

FIG. 4 is a block diagram showing a functional configuration of a user terminal;

FIG. 5 is a ladder chart showing medical data registration processing;

FIG. 6 is a ladder chart showing patient application use registration processing;

FIG. 7 is an example of a new registration screen displayed on the user terminal;

FIG. 8 is an example of an authentication code input screen displayed on the user terminal;

FIG. 9 is an example of a registration completion screen displayed on the user terminal;

FIG. 10 is an example of a facility list screen displayed on the user terminal;

FIG. 11 is an example of a hospital facility information screen displayed on the user terminal;

FIG. 12 is a ladder chart showing medical data registration processing in a modification example; and

FIG. 13 is a ladder chart showing patient application use registration processing in the modification example.

### DETAILED DESCRIPTION OF EMBODIMENTS

Hereinafter, embodiments of the present invention will be described with reference to the drawings. Though the following embodiments are provided with various technical limitations to implement the present invention, the scope of the invention is not limited to the disclosed embodiments or the illustrated examples.

<<Configuration of Medical Data Sharing System>>

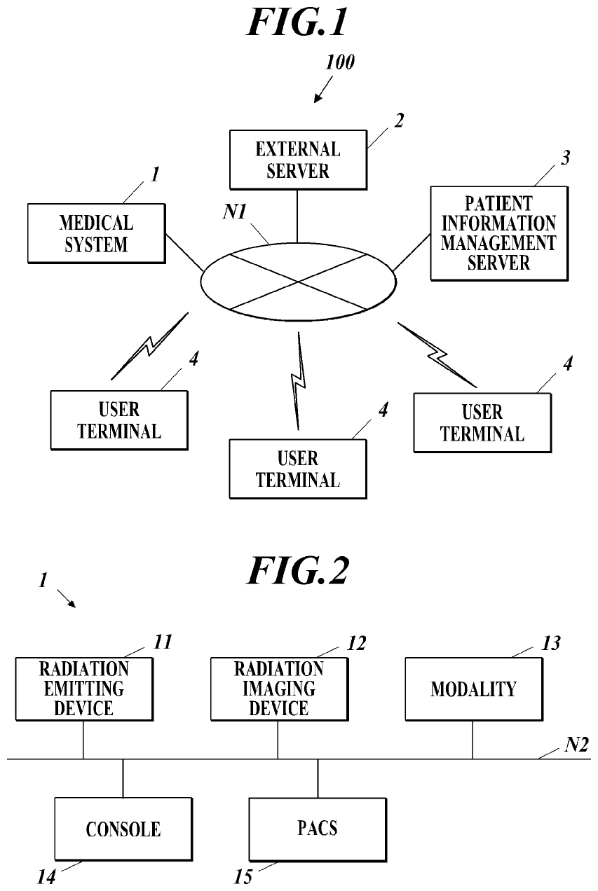

First, the configuration of one or more embodiments will be described with reference to FIG. 1. FIG. 1 is a schematic configuration view showing a medical data sharing system 100 in one or more embodiments.

In the medical data sharing system 100 in one or more embodiments, medical information of a patient (for example, information such as test results) which is obtained in a medical system 1 (to be described later) introduced to a hospital facility is viewed through a patient application 421 (to be described later) which is installed in a user terminal 4 (to be described later) used by the patient. That is, the medical data sharing system 100 is a system that enables sharing the medical information of the patient between the hospital facility and the patient.

As shown in FIG. 1, the medical data sharing system 100 includes a medical system 1, an external server 2, a patient information management server 3 and user terminals 4. The medical system 1, the external server 2, the patient information management server 3 and the user terminals 4 are connected so as to be able to communicate information with each other via a communication network N1.

The medical system 1 is introduced into a facility such as a hospital and a clinic, and enables a doctor to perform from tests to diagnosis alone. Though FIG. 1 shows only the medical system 1 which is introduced into a hospital (for example, hospital X), the number of medical systems 1 is not particularly limited.

The external server 2 is a server capable of freely storing and sharing data to be linked with other medical facilities and a patient information management server 3 to be described later, for example.

The patient information management server 3 is a server that manages information (for example, medical data) regarding a patient having an account registered in a patient application 421 (to be described later). That is, the patient information management server 3 manages the medical data of the patient or the like that can be shared with the hospital facility, and allows the patient to view the medical data of the patient or the like by logging in to the patient application 421 via the user terminal 4.

The user terminal 4 is a mobile terminal of the patient using the above-mentioned hospital X or the like into which the medical system 1 is introduced. The user terminal 4 is a smartphone or a portable phone, for example. Though FIG. 1 shows three user terminals 4, the number of user terminals 4 is not particularly limited.

The communication network N1 is the Internet, for example. However, the communication network N1 may be other networks.

<Configuration of Medical System>

Next, the functional configuration of medical system 1 will be described with reference to FIG. 2. FIG. 2 is a block diagram showing the functional configuration of the medical system 1.

As shown in FIG. 2, the medical system 1 includes a radiation emitting device 11, a radiation imaging device 12, a modality 13, a console 14 and a Picture Archiving and Communication System (PACS) 15. The medical system 1 is connected to a Hospital Information System (HIS) and a Radiology Information System (RIS) not shown in the drawings. These can communicate with each other via a communication network N2 (Local Area Network (LAN)), a Wide Area Network (WAN), the Internet, or the like.

The radiation emitting device 11 includes a generator that applies a voltage corresponding to radiation emitting conditions (tube voltage, tube current, emitting time (mAs value) and the like) which were set in advance in response to the operation of an irradiation instruction switch, a radiation source that generates radiation (for example, X ray) of a dose corresponding to the applied voltage in response to application of the voltage from the generator, and the like. The radiation emitting device 11 repeatedly emits pulsed radiation multiple times for a short time (for example, 15 times for one second). The radiation emitting device 11 may continuously emit radiation, not repeatedly emit pulsed radiation.

Though not shown in the drawings, the radiation imaging device 12 includes a substrate, scanning circuits, a reading circuit, a controller, an output section and the like. On the substrate, pixels including radiation detection elements that generate electric charges corresponding to the dose in response to reception of radiation and switch elements that accumulate and release electric charges are arranged two-dimensionally (in a matrix). The scanning circuits switch on/off of the respective switch elements. The reading circuit reads out the amounts of electric charges released from the respective pixels as signal values. The controller generates a radiographic image from the multiple signal values read by the reading circuit. The output section outputs data of the generated radiographic image and the like to the outside in a wired or wireless manner. The radiation imaging device 12 repeatedly generates the radiographic image corresponding to the emitted radiation multiple times in a short time (for example, 15 times for one second) in synchronization with the timing of emitting radiation from the radiation emitting device 11.

The modality 13 is an electrocardiogram, an ultrasonic diagnostic device, or an endoscopy inspection device, for example.

The console 14 is configured by a PC (personal computer), a portable terminal, a dedicated device, or the like. The console 14 can set various imaging conditions of the radiation emitting device 11 and the radiation imaging device 12 (for example, conditions regarding a subject such as a site to be imaged, tube voltage and tube current, conditions regarding emission of radiation such as emitting time) on the basis of the imaging order from other devices or systems (RIS and the like), operations by the user and the like.

The PACS 15 executes archiving, viewing, and management of radiographic images obtained by the radiation imaging device 12.

<Configuration of Patient Information Management Server>

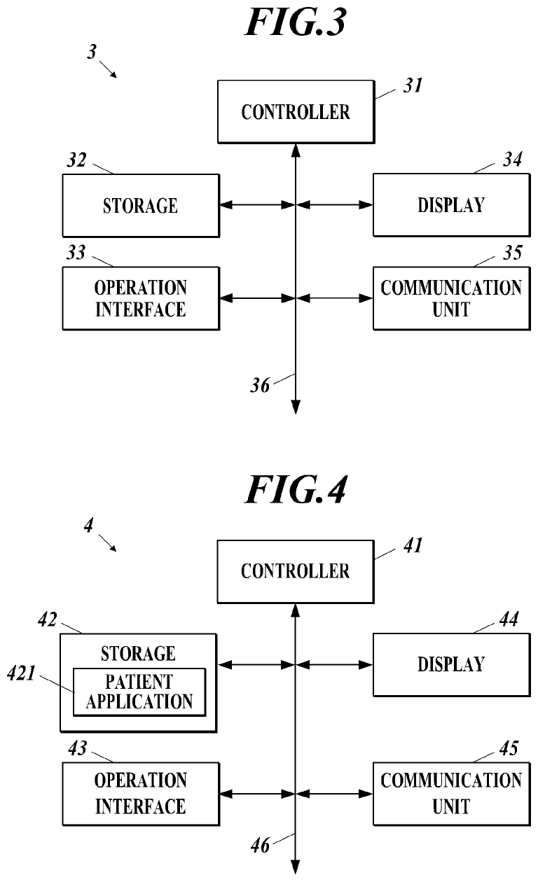

The functional configuration of the patient information management server 3 will be described with reference to FIG. 3. FIG. 3 is a block diagram showing the functional configuration of the patient information management server 3.

As shown in FIG. 3, the patient information management server 3 includes a controller 31 (hardware processor), a storage 32, an operation interface 33, a display 34, a communication unit 35, and a bus 36. The components of the patient information management server 3 are connected via the bus 36.

The controller 31 includes a CPU (Central Processing Unit) and a memory which are not shown in the drawings. The CPU implements various instructions stored in the storage 32 and executes predetermined calculations and controls of the components. The memory serves as a working area at the time of implementing the instructions. The controller 31 executes various types of processing in cooperation with the instructions stored in the storage 32.

The storage 32 is configured by including a non-volatile semiconductor memory or the like. The storage 32 stores system programs or instructions and application programs or instructions executed by the controller 31, data necessary to execute the instructions, and the like.

The operation interface 33 includes a key input section such as a keyboard, and a pointing device such as a mouse. The operation interface 33 receives a key operation input and a positional operation input from the user, and outputs the operation information to the controller 31. The controller 31 receives the input operation of the user on the basis of the information transmitted from the operation interface 33.

The display 34 is configured by including an LCD (Liquid Crystal Display), an EL (Electro Luminescence) display or the like, and performs various types of display on the screen in accordance with the display control signal from the controller 31.

The communication unit 35 is configured by including a modem, a router, a network card, or the like. The communication unit 35 communicates with external devices connected via the communication network N1.

<Configuration of User Terminal>

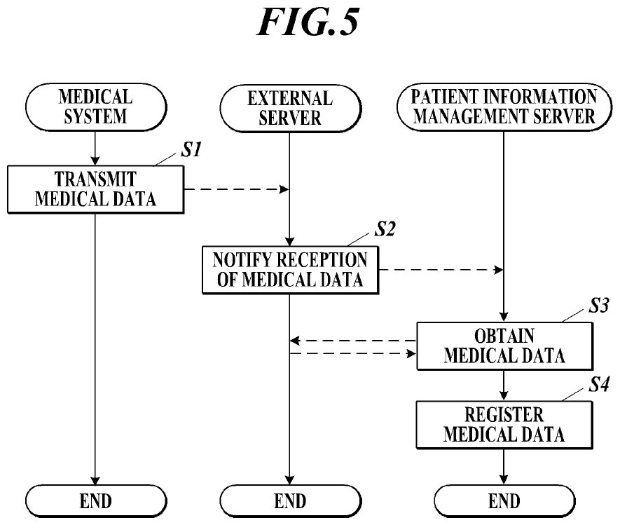

The functional configuration of the user terminal 4 will be described with reference to FIG. 4. FIG. 4 is a block diagram showing the functional configuration of the user terminal 4.

As shown in FIG. 4, the user terminal 4 includes a controller 41, a storage 42, an operation interface 43, a display 44, a communication unit 45, and a bus 46. The components of the user terminal 4 are connected via the bus 46.

The controller 41 includes a CPU (Central Processing Unit) and a memory which are not shown in the drawings. The CPU implements various instructions stored in the storage 42 and executes predetermined calculations and controls of the components. The memory serves as a working area at the time of implementing the instructions. The controller 41 executes various types of processing in cooperation with the instructions stored in the storage 42.

The storage 42 is configured by including a non-volatile semiconductor memory or the like. The storage 42 stores system programs or instructions and application programs or instructions (for example, patient application 421) executed by the controller 41, data necessary to execute the instructions, and the like.

The operation interface 43 includes various function keys. The operation interface 43 receives a pressing input of each key by the user (patient), and outputs the operation information to the controller 41. The operation interface 43 also includes a touch panel which has transparent electrodes arranged in a grid pattern so as to cover the surface of the display 44. The operation interface 43 detects the position which was pressed by a finger, a touch pen or the like, and outputs the positional information as an operation information to the controller 41.

The display 44 is configured by including an LCD (Liquid Crystal Display) or the like, and performs various types of display on the screen in accordance with the display control signal from the controller 41.

The communication unit 45 is wirelessly connected to the communication network N1, and communicates with external devices connected to the communication network N1.

<<Operation of Medical Data Sharing System>>

Next, the operation of medical data sharing system 100 will be described.

<Medical Data Registration Processing>

FIG. 5 is a ladder chart showing medical data registration processing executed in the medical data sharing system 100.

When a doctor in the above-mentioned hospital X explains the patient application 421 to a patient (for example, Mr. A), the medical data registration processing is performed in response to a trigger that the patient wishes to use the patient application 421. When the above patient wishes to use the patient application 421, the doctor gives, to the patient, a pamphlet provided with a two-dimensional registration code of the patient application 421.

As shown in FIG. 5, in the medical system 1, the medical data of the patient who wishes to use the patient application 421 is transmitted to the external server 2 via the operation by the above doctor (step S1). The medical data is, for example, a radiographic image of the patient which was captured at the time of medical examination, and displayed on a display device (not shown in the drawings) when the diagnosis result is explained to the patient.

When the medical data is received from the medical system 1, the external server 2 notifies the patient information management server 3 that the medical data is received (step S2).

when the patient information management server 3 receives, from the external server 2, the notification that the medical data was received, the controller 31 obtains the medical data from the eternal server 2 (step S3). The controller 31 then registers the medical data obtained in step S3 in the storage 32 (step S4). To be specific, in a case where the patient related to the medical data has already registered for the patient application 421 when the medical data is obtained in step S3, the controller 31 registers the medical data in the storage 32 so as to be associated with the patient information of the patient. On the other hand, in a case where the patient related to the medical data has not yet registered for the patient application 421 when the medical data is obtained in step S3, the controller 31 once stands by as for the association processing of the medical data with the patient information. When the patient related to the medical data has completed registration for the patient application 421, the controller 31 executes the work of associating the patient data of the medical system 1 with the information of the patient information management server 3 using the patient ID included in the medical data as a key, and registers the medical data in the storage 32.

The medical data registration processing then ends.

<Patient Application Use Registration Processing>

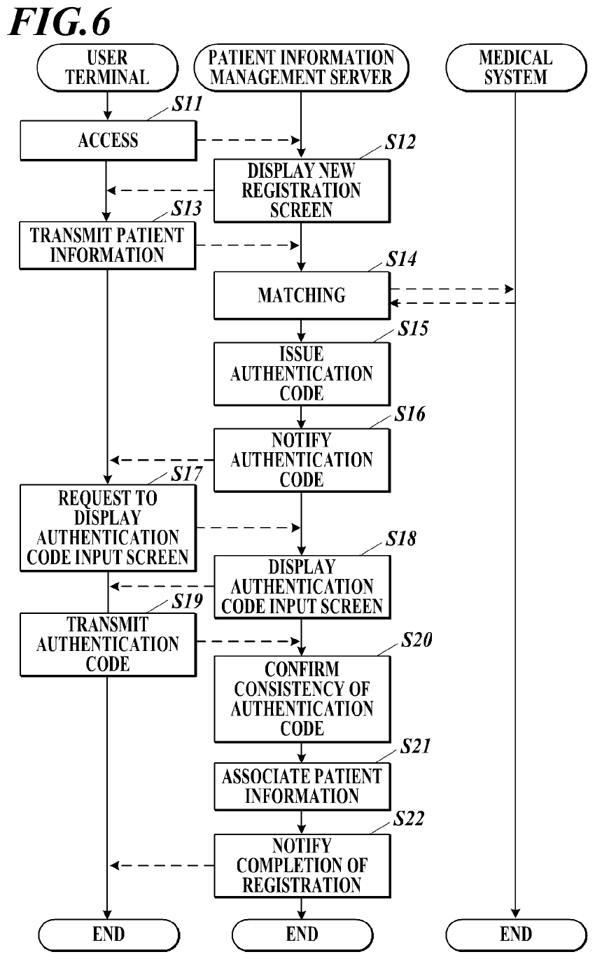

FIG. 6 is a ladder chart showing patient application use registration processing executed in the medical data sharing system 100.

After the patient who wishes to use the patient application 421 reads the two-dimensional registration code provided to the pamphlet which was given from the doctor of hospital X with the user terminal 4, when the patient accesses the URL (Uniform Resource Locator) which was converted from the two-dimensional registration code by the operation from the operation interface 43 (step S11), in the patient information management server 3, the controller 31 transmits display data for displaying a new registration screen via the communication unit 35 to the user terminal 4 (step S12).

In the user terminal 4, a new registration screen 51 is displayed on the display 44.

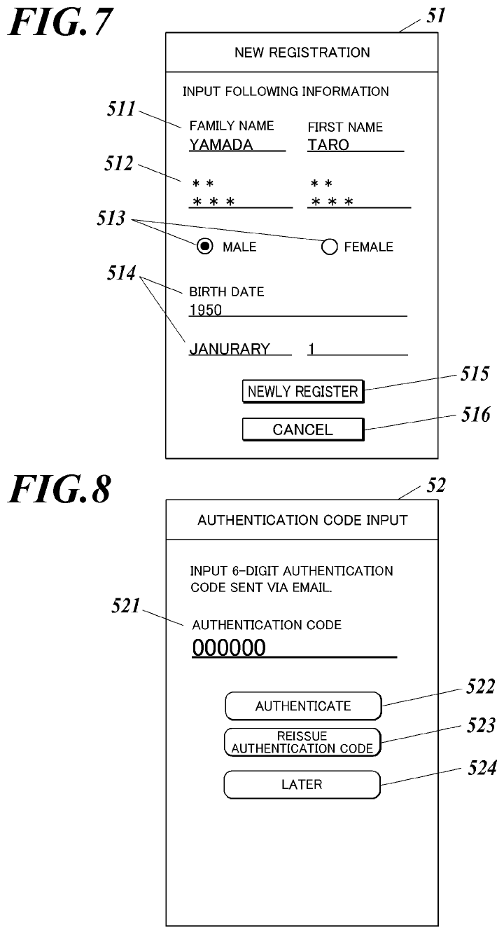

FIG. 7 shows an example of the new registration screen 51 displayed on the display 44. The new registration screen 51 includes a name (kanji) input region 511, a name (kana) input region 512, gender selection buttons 513, birth date input regions 514, a new registration button 515, a cancel button 516, and the like.

In the name (kanji) input region 511, the name (kanji) of the patient is input.

In the name (kana) input region 512, the name (kana) of the patient is input.

The gender selection buttons 513 are buttons for selecting the gender of the patient.

In the birth date input regions 514, the birth date of the patient is input.

The new registration button 515 is a button for instructing to transmit the information which was input in the above name (kanji) input region 511, the name (kana) input region 512, the gender selection buttons 513, and the birth date input regions 514, that is, the patient information to the patient information management server 3.

The cancel button 516 is a button for canceling the input of patient information.

In the new registration screen 51, when the patient performs touch operation to the new registration button 515 by the operation from the operation interface 43 after inputting the patient information, the controller 41 transmits the patient information which was input in the new registration screen 51 via the communication interface 45 to the patient information management server 3 (step S13).

In the patient information management server 3, when the patient information is received by the communication unit 35, the controller 31 executes matching of the patient information (step S14). To be specific, the controller 31 accesses a hospital information system (not shown in the drawings) connected to the medical system 1 via the communication unit 35. The controller 31 then searches for the patient data that matches the patient information which was input on the user terminal 4, in the patient list of the hospital information system. When there is patient data matching the patient information in the patient list, the controller 31 obtains the patient data. In one or more embodiments, the patient data includes the examination ticket number, names (kanji name and kana name), gender, birth date, phone number, email address and the like.

In the patient information management server 3, the controller 31 issues a 6-digit authentication code (step S15). The authentication code issued in one or more embodiments is, for example, an authentication code which is temporarily issued with a set validity period and is in such a form that the patient is not identified from outside. The authentication code is generated on the basis of the patient data of the patient that is a target, for example. That is, the issued authentication code is a unique code associated with the patient.

In the patient information management server 3, the controller 31 notifies the authentication code issued in step S15 to the portable phone number included in the patient data obtained in step S14 by using an SMS (Short Message Service) (step S16). The controller 31 may notify the authentication code issued in step S15 to the email address included in the patient data obtained in step S14.

In the user terminal 4, when the authentication code is received by the communication unit 45, the controller 41 requests to display an authentication code input screen via the communication unit 45 to the patient information management server 3 by the operation from the operation interface 43 (step S17).

In the patient information management server 3, when the request to display the authentication code input screen is received by the communication unit 35, the controller 31 transmits display data for displaying the authentication code input screen via the communication unit 35 to the user terminal 4 (step S18).

On the user terminal 4, the authentication code input screen 52 is displayed on the display 44.

FIG. 8 shows an example of the authentication code input screen 52 displayed on the display 44. The authentication code input screen 52 includes an authentication code input region 521, an authentication button 522, a reissue button 523, a cancel (later) button 524, and the like.

In the authentication code input region 521, the authentication code notified by the patient information management server 3 is input.

The authentication button 522 is a button for instructing to transmit the authentication code which was input in the authentication code input region 521 to the patient information management sever 3.

The reissue button 523 is a button for requesting the patient information management server 3 to reissue the authentication code.

The cancel (later) button 524 is a button which is operated when the work of inputting the authentication code is performed later.

On the authentication code input screen 52, when the patient performs touch operation of the authentication button 522 by the operation from the operation interface 43 after inputting the authentication code, the controller 41 transmits the authentication code input on the authentication code input screen via the communication unit 45 to the patient information management server 3 (step S19).

In the patient information management server 3, when the authentication code is received by the communication unit 35, the controller 31 confirms consistency between the received authentication code and the authentication code issued in step S15 (step S20). When it is confirmed that the received authentication code is consistent with the authentication code issued in step S15, in the patient information management server 3, the controller 31 associates the patient information received from the user terminal 4, the patient data obtained from the hospital information system connected to the medical system 1, and the medical data of the corresponding patient with each other, and stores them in the storage 32 (step S21).

In the patient information management server 3, the controller 31 notifies that the registration for using the patient application 421 has completed via the communication unit 35 to the user terminal 4 (step S22).

In the user terminal 4, the registration completion screen 53 is displayed on the display 44.

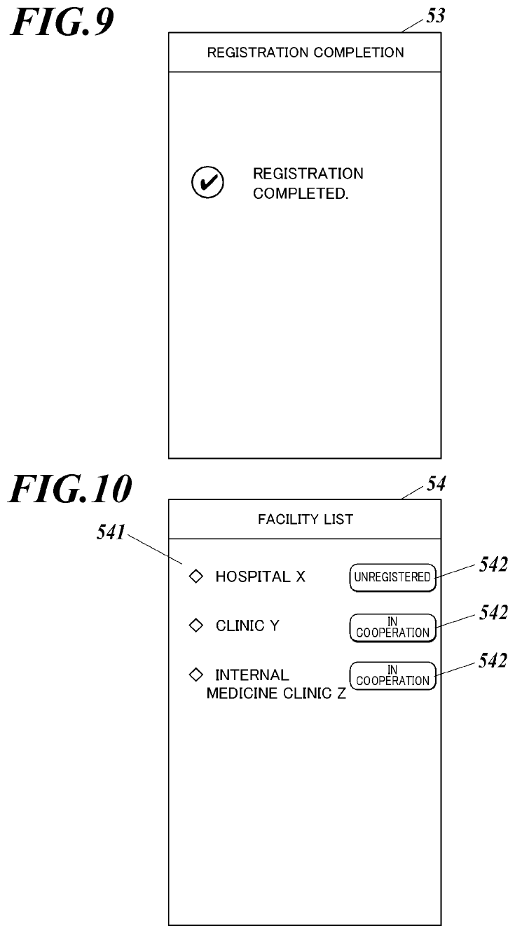

FIG. 9 shown an example of the registration completion screen 53 displayed on the display 44. On the registration completion screen 53, a message indicating the completion of registration for using the patient application 421 (for example, “Registration Completed.”) is displayed.

The patient application use registration processing then ends.

On the authentication code input screen 52, when the patient performs the registration for using the patient application 421 after performing the touch operation of the cancel (later) button 524 by the operation from the operation interface 43, in the user terminal 4, the facility list screen 54 is displayed on the display 44 by the operation from the operation interface 43.

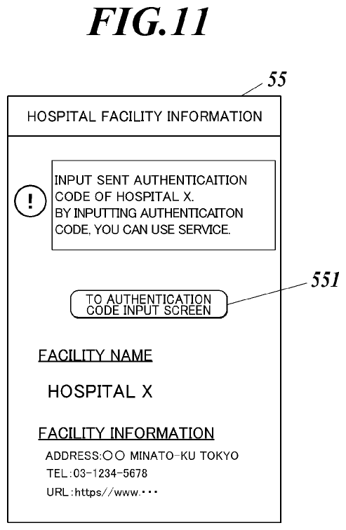

FIG. 10 shows an example of the facility list screen 54 displayed on the display 44. In the facility list screen 54, a list 541 of hospital facilities for which registration for using the patient application 421 is completed and hospital facilities for which registration for using the patient application 421 is not completed is displayed. The status 542 corresponding to each of the hospital facilities is also displayed. The status 542 has two types of “in cooperation” and “unregistered”. When the registration for using the patient application 421 is completed by the patient application use registration processing, the status 542 of the corresponding hospital facility is displayed as “in cooperation”. On the other hand, when the registration for using the patient application 421 is not completed by the patient application use registration processing, the status 542 of the corresponding hospital facility is displayed as “unregistered”.

The facility list screen 54 shown in FIG. 10 shows that the status 542 of the hospital X is in the “unregistered” state, that is, the state in which the registration for using the patient application 421 regarding the hospital X is not completed. When the patient selects the hospital X by the operation from the operation interface 43, in the user terminal 4, the hospital facility information screen 55 is displayed on the display 44. When the status 542 of the hospital X is in the state of “in cooperation” and the patient selects the hospital X by the operation from the operation interface 43, in the user terminal 4, there is displayed a viewing screen (not shown in the drawings) allowing the user to view the medical data and the like of the hospital X.

FIG. 11 shows an example of the hospital facility information screen 55 displayed on the display 44. In the hospital facility information screen 55, information regarding the selected hospital facility (for example, hospital X) is displayed, and the screen shift button 551 for shifting to the authentication code input screen 52 is provided. In the hospital facility information screen 55, a message regarding inputting the authentication code is displayed above the screen shift button 551. When the patient performs touch operation to the screen shift button 551 by the operation from the operation interface 43, in the user terminal 4, the authentication code input screen 52 is displayed on the display 44, and enables the patient to restart the patient application use registration processing.

As described above, the patient information management server 3 confirms whether at least part of patient information (first patient information) obtained from the user terminal 4 which is used by the patient matches at least part of patient data (second patient information) obtained from the medical facility. The patient information management server 3 notifies the user terminal 4 of the authentication code (authentication information) of the patient which is generated in response to confirming that the at least part of the patient information matches the at least part of the patient data. When authentication code (related authentication information related to the authentication information) is received from the user terminal 4 which received the authentication code, the patient information management server 3 associates the patient information with the patient data.

Accordingly, according to the patient information management server 3, at the time of account registration for the patient application 421, it is possible to verify identity without face-to-face contact with the patient by using the user terminal 4 which is used by the patient. As a result, the account registration for the patient application 421 can be performed even on the way home from the hospital facility, after getting home, and the like, and thus, it is possible to save the trouble of visiting the hospital again for registering the account.

When the patient information management server 3 notifies the authentication code (authentication information) of the patient to the user terminal 4, the patient information management server 3 determines the transmission information (phone number) for transmitting the authentication code of the patient to the user terminal 4, and transmits the authentication code of the patient to the user terminal 4 by using the transmission information. Thus, it is possible to appropriately notify the authentication code to the user terminal 4.

Since the patient information management server 3 generates the transmission information from the patient data (second patient information), it is possible to efficiently generate the transmission information.

When the patient information management server 3 associates the patient information with the patient data, the patient information management server 3 confirms whether the notified authentication code and the received authentication code correspond to each other in response to reception of the authentication code (related authentication information) from the user terminal 4. If it is confirmed that the notified authentication code and the received authentication code correspond to (consistent with) each other, the patient information management server 3 associates the patient information with the patient data. Thus, it is possible to appropriately execute the association.

The patient information management server 3 obtains the patient data (second patient information) from the hospital information system via the medical system 1. Accordingly, according to the patient information management server 3, it is possible to suppress the cost related to the storage in the server by obtaining the necessary patient data each time the patient data is needed.

### Modification Example

A modification example in the above embodiments will be described. The same reference numerals are provided to the same components as those of the above embodiments and explanation thereof is omitted.

<Medical Data Registration Processing>

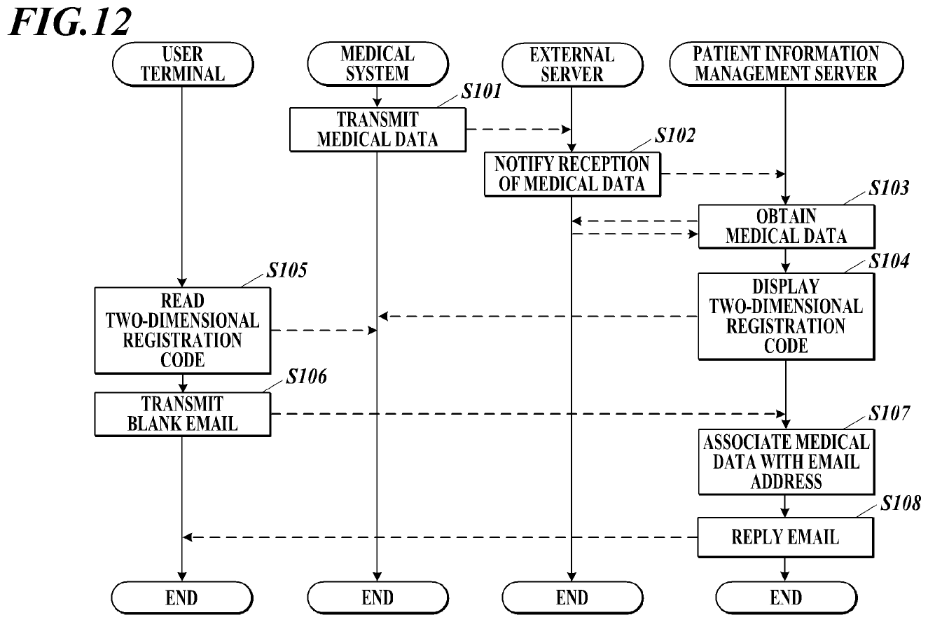

FIG. 12 is a ladder chart showing medical data registration processing in a modification example.

In steps S101 to S103 of the medical data registration processing in the modification example, since the same processes as those of steps S1 to S3 of the medical data registration processing in one or more embodiments are performed, the explanation for these steps is omitted, and the processes after the step S104 will be described.

As shown in FIG. 12, in the patient information management server 3, the controller 31 transmits display data for displaying a two-dimensional registration code via the communication unit 35 to the medical system 1 after obtaining the medical data (step S104). This two-dimensional registration code is obtained by coding an email address (email address managed by the patient information management server 3) accompanied by a medical data ID that enables identifying the medical data transmitted to the patient information management server 3 and a hospital facility ID that enables identifying the hospital facility which is the source of creating the medical data.

In the medical system 1, the above two-dimensional registration code is displayed on a personal computer used by the doctor at the time of medical interview with the patient. At this time, the two-dimensional registration code is read out by the user terminal 4 (step S105). The controller 41 of the user terminal 4 transmits a blank email to the email address decoded from the two-dimensional registration code (step S106). Both of the work of reading out the two-dimensional registration code and the work of transmitting the blank email by the user terminal 4 are watched by the doctor. The doctor watches both of the works because it is necessary to reissue the two-dimensional registration code if a transmission error has occurred due to the work of transmitting the blank email being executed after the medical interview is finished and the patient exits the examination room, and reissuing the two-dimensional registration code is troublesome for both of the hospital facility and the patient.

In the patient information management server 3, when the above blank email is received by the communication unit 35, the controller 31 associates the email address of the user terminal 4 which is the transmission source of the blank email with the medical data ID and the hospital facility ID accompanying the two-dimensional code, and stores them in the storage 32 (step S107). The controller 31 transmits a reply email via the communication unit 35 to the user terminal 4 which is the transmission source of the blank email (step S108). The URL for accessing the registration site of the patient application 421 is attached to the reply email.

The medical data registration processing then ends.

<Patient Application Use Registration Processing>

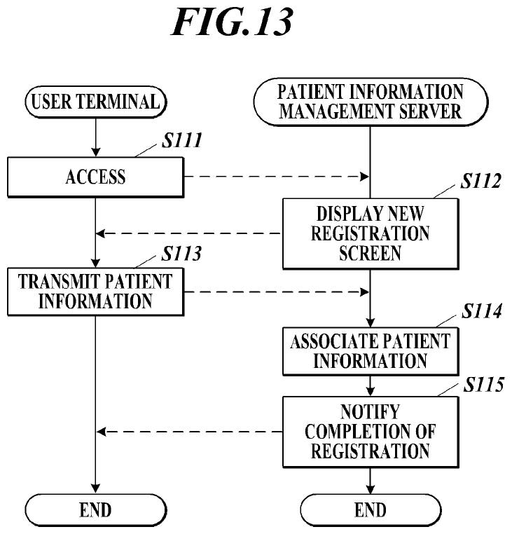

FIG. 13 is a ladder chart showing patient application use registration processing in a modification example.

When the patient who wishes to use the patient application 421 accesses the URL attached to the reply email from the patient information management server 3 by the operation from the operation interface 43 in the user terminal 4 (step S111), in the patient information management server 3, the controller 31 transmits the display data for displaying the new registration screen 51 via the communication unit 35 to the user terminal 4 (step S112).

On the new registration screen 51, when the patient performs touch operation to the new registration button 515 by the operation from the operation interface 43 after inputting the patient information, the controller 41 transmits the patient information input on the new registration screen 51 via the communication unit 45 to the patient information management server 3 (step S113).

In the patient information management server 3, when the patient information is received by the communication unit 35, the controller 31 associates the patient information received from the user terminal 4, the email address of the user terminal 4, and the corresponding medical data ID and the hospital facility ID with each other, and stores them in the storage 32 (step S114).

In the patient information management server 3, the controller 31 notifies that the registration for using the patient application 421 has completed via the communication unit 35 to the user terminal 4 (step S115).

The patient application use registration processing then ends.

As described above, according to the patient information management server 3 in the modification example, it is possible to omit identity verification of the patient performed in the patient application use registration processing as in one or more embodiments, by associating the email address of the user terminal 4 used by the patient with the medical data ID and the hospital facility ID in the medical data registration processing.

<<Others>>

Though the present invention has been described on the basis of the above embodiments, the present invention is not limited to the above embodiments and the modification example, and modifications can be made as needed within the scope of the present invention.

For example, in one or more embodiments, the name, the gender, and the birth date are the target of matching in the matching (step S14) of the patient application use registration processing. However, the target of matching is not limited to these items of information. For example, the examination ticket number may be the target of matching by having a configuration to input the examination ticket number of the patient in the new registration screen 51.

For example, the email address and the phone number of the user terminal 4 used by the patient may be the target of matching by having a configuration to input the email address and the phone number in the new registration screen 51. In such a case, for example, when the input email address does not match the same information of the patient data, inputting the email address again may be urged by notifying the patient that the input email address does not match the same information of the patient data. Thus, it is possible to prevent the authentication code and the like from being transmitted to a wrong address.

In one or more embodiments, in the authentication code transmission processing of patient application use registration processing (step S19), the 6-digit authentication code notified from the patient information management server 3 is transmitted. As a code (related authentication information) related to the authentication code, for example, the number obtained by multiplying all the numbers in respective digits of the authentication code may be transmitted.

The above description discloses an example of using a hard disk, semiconductor nonvolatile memory, or the like as a computer readable medium of instructions according to one or more embodiments of the present invention. However, the medium is not limited to this example. As other computer readable medium, a portable storage medium such as a CD-ROM can be applied. A carrier wave can also be applied as a medium providing the program or instruction data according to one or more embodiments of the present invention via a communication line.

As for the other detailed configurations and detailed operations of the devices forming the medical data sharing system 100, modifications can be made as needed within the scope of the present invention. For example, the patient information management server 3 may be a system with PACS functions, a system with console functions in addition to the PACS functions, or a system with electronic medical chart functions. The instructions according to one or more embodiments of the present invention can be executed even in these systems.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A non-transitory computer readable storage medium storing instructions that cause a computer to execute:
confirming whether at least part of first patient information matches at least part of second patient information, wherein the first patient information is obtained from a user terminal used by a patient and the second patient information is obtained from a medical facility;
notifying the user terminal of authentication information of the patient, wherein the authentication information is generated upon confirming that the at least part of the first patient information matches the at least part of the second patient information; and
associating the first patient information with the second patient information upon reception of related authentication information relating to the authentication information from the user terminal to which the authentication information has been notified.

2. The storage medium according to claim 1, wherein, in the notifying, the instructions cause the computer to execute:
determining transmission information for transmitting the authentication information of the patient to the user terminal; and
transmitting the authentication information of the patient to the user terminal by using the determined transmission information.

3. The storage medium according to claim 2, wherein, in the determining, the transmission information is determined from the second patient information.

4. The storage medium according to claim 1, wherein, in the associating, the instructions cause the computer to execute:
confirming whether the notified authentication information corresponds to the related authentication information upon reception of the related authentication information from the user terminal; and
associating the first patient information with the second patient information upon confirming that the notified authentication information corresponds to the related authentication information.

5. The storage medium according to claim 1, wherein the authentication information is a unique code related to the patient.

6. The storage medium according to claim 5, wherein the authentication information is generated from the second patient information.

7. The storage medium according to claim 5, wherein a validity period is set for the authentication information.

8. The storage medium according to claim 5, wherein the authentication information is an authentication code in a form that the patient is not identified from outside.

9. The storage medium according to claim 1, wherein the instructions further cause the computer to execute obtaining the second patient information from an external device.

10. A non-transitory computer readable storage medium storing instructions that cause a computer to execute:
obtaining first patient information from a user terminal used by a patient;
obtaining second patient information from a medical facility;
confirming whether at least part of the first patient information matches at least part of the second patient information;
generating authentication information of the patient upon confirming that the at least part of the first patient information matches the at least part of the second patient information;
determining transmission information for transmitting the authentication information to the user terminal;
transmitting the authentication information to the user terminal by using the determined transmission information; and
associating the first patient information with the second patient information upon reception of related authentication information relating to the authentication information from the user terminal.

11. A patient information management device comprising:
a hardware processor that:
confirms whether at least part of first patient information matches at least part of second patient information, wherein the first patient information is obtained from a user terminal used by a patient and the second patient information is obtained from a medical facility;
notifies the user terminal of authentication information of the patient, wherein the authentication information is generated upon confirming that the at least part of the first patient information matches the at least part of the second patient information; and
associates the first patient information with the second patient information upon reception of related authentication information relating to the authentication information from the user terminal to which the authentication information has been notified.

12. A patient information management device comprising:
a hardware processor that:
obtains first patient information from a user terminal used by a patient;
obtains second patient information from a medical facility;
confirms whether at least part of the first patient information matches at least part of the second patient information;
generates authentication information of the patient upon confirming that the at least part of the first patient information matches the at least part of the second patient information;
determines transmission information for transmitting the authentication information to the user terminal;
transmits the authentication information to the user terminal by using the determined transmission information; and
associates the first patient information with the second patient information upon reception of related authentication information relating to the authentication information from the user terminal.
